(19) United States
(12) Patent Application Publication
(10) Pub. No.: US 2025/0270502 A1
(43) Pub. Date: Aug. 28, 2025

(54) SYSTEMS AND METHODS FOR RAPID LIQUID BIOPRINTING

(71) Applicant: Innocent Meat GmbH, Papendorf (DE)

(72) Inventor: Philipp Drescher, Rostock (DE)

(73) Assignee: Innocent Meat GmbH, Papendorf (DE)

(21) Appl. No.: 19/060,824

(22) Filed: Feb. 24, 2025

Related U.S. Application Data
(60) Provisional application No. 63/557,613, filed on Feb. 26, 2024.

(51) Int. Cl. B33Y 30/00; B33Y 10/00; B33Y 70/00; C12N 5/00

(52) U.S. Cl. CPC C12N 5/0068; B33Y 10/00; B33Y 30/00; B33Y 70/00; C12N 2533/74; C12N 2537/10

(57) ABSTRACT

Methods for fabricating three-dimensional (3D) constructs from a mixture of cells, culture medium, and a scaffold material with a controlled calcium chloride concentration is disclosed. The process involves positioning a nozzle within a gel composed of alginate solution. The nozzle is then moved within the gel, releasing the cell-media-scaffold mixture in a controlled pattern. As the mixture is deposited, it becomes surrounded by the gel, which provides structural support until it solidifies through the cross-linking reaction between the calcium chloride in the cell-media-scaffold mixture and the alginate solution. This results in the formation of a solid 3D structure that incorporates the cells and their supporting scaffold. The presented method offers a versatile platform for generating intricate cell-based 3D constructs with precise control over their composition and structure. This method can be used to produce cultivated meat with complex 3D structures as well as organelles and fully functional organs.

### CROSS REFERENCE TO RELATED APPLICATIONS

This application is a U.S. Non-Provisional Utility patent application entitled, “SYSTEMS AND METHODS FOR RAPID LIQUID BIOPRINTING” that claims priority to U.S. Provisional Patent Application No. 63/557,613 filed on Feb. 26, 2024, entitled, “SYSTEMS AND METHODS FOR RAPID LIQUID BIOPRINTING” the contents of which are hereby fully incorporated by reference.

### FIELD OF THE INVENTION

The present invention relates to methods for fabricating three-dimensional (3D) constructs from a mixture of cells, culture medium, and a scaffold material with a controlled calcium chloride concentration. The process involves positioning a nozzle within a gel composed of alginate solution. The nozzle is then moved within the gel, releasing the cell-media-scaffold mixture in a controlled pattern. As the mixture is deposited, it becomes surrounded by the gel, which provides structural support until it solidifies through the cross-linking reaction between the calcium chloride in the cell-media-scaffold mixture and the alginate solution. This results in the formation of a solid 3D structure that incorporates the cells and their supporting scaffold. The presented method offers a versatile platform for generating intricate cell-based 3D constructs with precise control over their composition and structure. This method can be used to produce cultivated meat with complex 3D structures as well as organelles and fully functional organs. The cell-media-scaffold mixture can be composed of different cell types such as muscle tissue, fibroblasts or endothelial cells etc.

### BACKGROUND OF THE INVENTION

Bioreactors first became prevalent in the 20^{th} century. Initially, the bioreactors were limited to being used for bacterial cell production, for small scale tissue culture propagation, and for development of vaccines. In the 1990s, bioreactor use expanded to being able to be used for more complex eukaryotic cells like mammalian cells, insect cells, and complex plant cells. More recently, bioreactors have been used to grow cultivated meat and in 2013 a Dutch scientist by the name of Mark Prost unveiled the first cultivated meat patty. Not long thereafter, cultivated meat companies were developed in an attempt to commercialize cultivated meat and/or cultivated plant-based meat-like products.

Alginate (or alginic acid) was first described and extracted from seaweed in the 1880s by the British chemist E.C.C. Stanford. Commercial production of alginate started in earnest in the 1920s, and it was and has been used in a number of different industries. For example, in the mid 1950s alginate was being used in the food industry in a number of products, particularly for its use as a food stabilizer. Alginate containing products have also been used in the textile and paper industries. Around 1933, it was discovered that alginate could be used for its microencapsulation properties as tumor cells were microencapsulated and inserted into the abdomen of guinea pigs without being rejected. More recently, alginate has been implicated in stem cell related research technologies. For example, the relative ease with which alginate hydrogels can be produced has made its use ideal for immune-isolated stem cell delivery systems. Alginate has been used for wound healing and for regenerating human tissue.

In the 1940s, two dimensional alginate based systems were developed for cell culture. Later, three dimensional alginate based systems were developed that allowed for more relevant drug development and testing, more realistic and controllable cell cultures that mimic tissues and organs, better systems for allowing cell growth, and more realistic models for immune system mimicry. Alginate hydrogels have been more recently developed that are ideal structures for cell immobilization.

### BRIEF SUMMARY OF THE INVENTION

The present invention relates to methods for fabricating three-dimensional (3D) constructs from a mixture of cells, culture medium, and a scaffold material with a controlled calcium chloride concentration. The process involves positioning a nozzle within a gel composed of alginate solution. The nozzle is then moved within the gel, releasing the cell-media-scaffold mixture in a controlled pattern. As the mixture is deposited, it becomes surrounded by the gel, which provides structural support until it solidifies through the cross-linking reaction between the calcium chloride in the cell-media-scaffold mixture and the alginate solution. This results in the formation of a solid 3D structure that incorporates the cells and their supporting scaffold. The presented method offers a versatile platform for generating intricate cell-based 3D constructs with precise control over their composition and structure. This method can be used to produce cultivated meat with complex 3D structures as well as organelles and fully functional organs. The cell-media-scaffold mixture can be composed of different cell types such as muscle tissue, fibroblasts or endothelial cells etc.

Different concentrations of alginate in the gel as well as calcium chloride in the media, or as a separate additive, can lead to different degrees of cross-linking and therefore adjustable solidification and mechanical strengths. The cell-media-scaffold mixture will be encapsulated by the surrounding alginate gel, causing a solidification on its outside while remaining semi-liquid on the inside, which functions as a temporary and local bioreactor environment. The semi-liquid condition of the cell-media-scaffold mixture keeps the cells within the scaffolds undisturbed, resulting in the final consumption of media components and the formation of the desired tissue structure.

### BRIEF DESCRIPTION OF THE SEVERAL VIEWS OF THE DRAWING

FIG. 1 depicts several drawings that show several containers of the present invention including a bioreactor wherein the several containers comprise a nozzle and alginate gel media comprising calcium chloride.

FIG. 2 depicts a cross-sectional view of the alginate based gel with the various layers that are generated by the alginate gel comprising calcium chloride and discharge from the nozzle.

### DETAILED DESCRIPTION OF THE INVENTION

This invention describes a method for fabricating three-dimensional (3D) constructs from a mixture of cells, culture medium, and a scaffold material with a controlled calcium chloride concentration. The process involves positioning a nozzle within a gel composed of alginate solution. The nozzle is then moved within the gel, releasing the cell-media-scaffold mixture in a controlled pattern. As the mixture is deposited, it becomes surrounded by the gel, which provides structural support until it solidifies through the cross-linking reaction between the calcium chloride in the cell-media-scaffold mixture and the alginate solution. This results in the formation of a solid 3D structure that incorporates the cells and their supporting scaffold. The presented method offers a versatile platform for generating intricate cell-based 3D constructs with precise control over their composition and structure. This method can be used to produce cultivated meat with complex 3D structures as well as organelles and fully functional organs. The cell-media-scaffold mixture can be composed of different cell types such as muscle tissue, fibroblasts or endothelial cells etc.

Different concentrations of alginate in the gel as well as calcium chloride in the media, or as a separate additive, can lead to different degrees of cross-linking and therefore adjustable solidification and mechanical strength. The cell-media-scaffold mixture will be encapsulated by the surrounding alginate gel, causing a solidification on its outside while remaining semi-liquid on the inside, which functions as a temporary and local bioreactor environment. The semi-liquid condition of the cell-media-scaffold mixture keeps the cells within the scaffolds undisturbed, resulting in the final consumption of media components and the formation of the desired tissue structure.

In an embodiment, the present invention relates to adding the calcium chloride in a manner that is controlled by a computer. In a variation, the computer is able to ascertain the spatial location of the nozzle so that it can ascertain if the nozzle has deposited calcium chloride or not in a given location. In a variation, the computer has a monitor that is able to display a three-dimensional map showing exactly where the nozzle has deposited calcium chloride. Thus, a user can automatically get a display of where the nozzle still needs to go to fill in the space (or voids) with calcium chloride (solution). The associated software necessary to ascertain if an entire volume of a solution has been covered is one embodiment of the present invention.

In an embodiment, sodium alginate is dissolved in a water solution so that it generates a viscous, clear solution wherein the sodium alginate is equally dispersed throughout the aqueous solution. The alginate solution may be partially cross-linked in order to improve the precise depositing of the cell-media-scaffold mixture, resulting in higher accuracy, precision and stability of the solidified structure. The sodium alginate solution may be placed in a bioreactor or in another container where cell growth is to take place. In a variation, when calcium chloride is introduced to the sodium alginate solution, a double replacement reaction occurs wherein the calcium ions replace the sodium ions (with sodium chloride as the other reaction product) thereby generating a cross-linked scaffold that comprises calcium alginate. Alginate (or alginic acid, depending on if cation salts are present) is a biopolymer formed of chains of polyuronic acids with homopolymeric blocks of (1,4) linked b-D-mannuronate and a-L-guluronate. Because the calcium is a di-cation (whereas sodium is a mono-cation), the replacement of sodium by calcium allows for three-dimensional cross-linking to occur. Ionic cross-linking between multivalent cations and alginate takes place instantaneously. The resulting calcium alginate provides a relatively solid matrix scaffold on which 3-dimensional cell growth can occur.

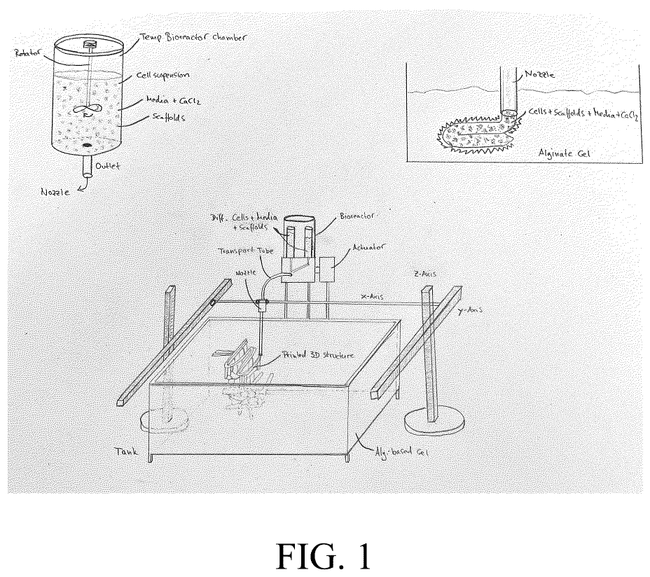

In an embodiment, and as shown in FIG. 1, a solution is prepared in a container that comprises a calcium chloride containing mix that also has cells, media and scaffolds present in it. In a variation, there may be a stirrer associated with the container that allows the calcium chloride and the cells to be evenly dispersed throughout the solution in the container and providing the cells with continuous access to nutrients and oxygen. There may also be a nozzle associated with the container that allows the solution inside the container to be dispersed through the nozzle into another container. In an embodiment, the nozzle may work by simple gravity and or the use of a mechanical feed, such as a screw extruder, to allow the solution in the container to pass through the nozzle. Alternatively, there may be positive pressure present in the container that allows the contents therein to be dispersed against a gradient. In this embodiment, even if the contents in the container (containing calcium chloride and cells) are being discharged into a viscous solution in another container, the positive pressure in the container allows the contents to be emptied. In a variation, the pressure may be variable to allow an equal dispersion of the contents thereof to be equally dispersed when put into another container.

As shown in the upper right-hand figure in FIG. 1, the nozzle is showing the output of cells, media, calcium chloride, and/or scaffold material through the nozzle into the alginate gel containing container. In a variation, the scaffold forms when the calcium chloride contacts the sodium alginate containing solution allowing for the formation of a three-dimensional calcium alginate gel matrix.

As shown in the bottom figure in FIG. 1, there may be an actuator associated with the nozzle and the container that allows the nozzle to be positioned at and/or moved to any position in a tank/container (for example, using x, y, and z coordinates). Thus, the nozzle cannot only be actuated so that it moves in longitudinal or lateral directions (i.e., horizontally in the x and y directions), but it can also move in a vertical direction (the z direction). This allows the nozzle to attain any position in a given container. The nozzle may be moved by a user, or alternatively, the nozzle may be moved by use of a computer program that allows one to access any position in the tank/container that comprises the alginate-based gel.

The nozzle is connected to a transport tube at one end and at a second end, the transport tube is operationally connected to a bioreactor or other container containing the cells, media, calcium chloride, and optionally scaffolding material. When these components leave the bioreactor and are deposited into the alginate solution, they have the effect of creating a printed 3D structure (see the bottom figure in FIG. 1).

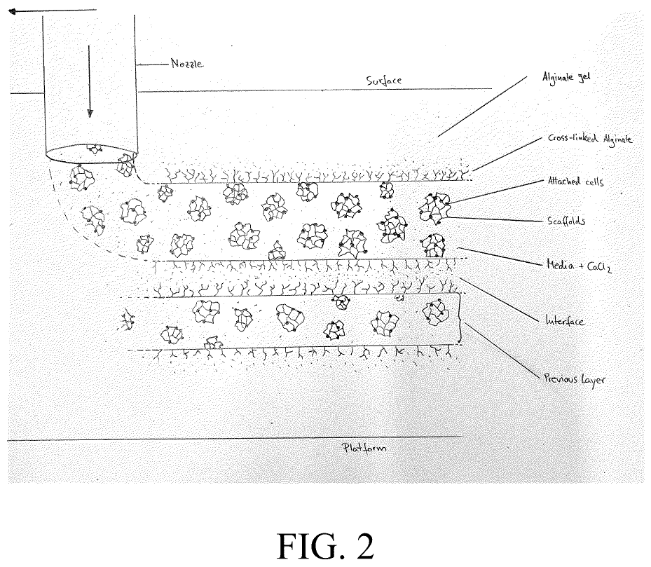

FIG. 2 shows a blow up of the nozzle that is dispensing components into the alginate gel containing container. As shown in FIG. 2, there may be present cross-linked alginate, scaffolds, with attached cells, media and calcium chloride (CaCl₂)), and an interface. As shown in FIG. 2, there may be a previous layer, which happens because the nozzle has previously passed over the region where the previous layer is formed. Thus, it should be apparent that as the nozzle continues its passage through the container, there are constantly new layers that are being deposited upon layers that have been previously formed (where the nozzle previously passed). As is shown in FIG. 2, there may be a cross-linked alginate layer with another layer that comprises scaffolding with cells attached to the scaffolds. The scaffolding is surrounded by media that comprises the calcium chloride. As shown in FIG. 2, there is also an interface layer, which is present between the scaffolding/cell/media layers. The interface layer is made up of cross-linked alginate and functions as a stable structure for the semi-liquid component.

There may also be one or more sensors operationally attached and/or associated with the nozzle that allows the nozzle to not only be able to map its position, but also allow the nozzle to “sense” the local conditions at its position. For example, there may be one or more sensors that is able to ascertain a temperature, dissolved oxygen, or a pH or other important conditions. The sensor may also be able to ascertain the number or concentration of cells in a given location by use of Doppler ultrasound wherein an ultrasound transducer is mounted on the outside of the bioreactor and emits a high frequency tone burst that occurs throughout the bioreactor container. In an embodiment, the number of concentration of cells may be determined by impedance cytometry. The sensors associated with the nozzle may also have optical (and magnification) abilities allowing a user to ascertain the shape of the three dimensional matrix in a given location in the container.

As disclosed herein, there may also be associated with the nozzle and the actuator the requisite software and or hardware necessary to accomplish the ability to move the nozzle appropriately in a container to deposit calcium chloride and cells.

For example, the present invention may include axis, actuators and a computer system, a method, and/or a computing device, that are already established and fully functional technologies for additive manufacturing. In a basic configuration of a computing device, the computing device may include one or more processors and a system memory. In some embodiments, the computing device may include one or more processors and a system memory. A memory bus may be used for communicating between the one or more processors and the system memory.

Depending on the desired configuration, the processor may be of any type, including, but not limited to, a microprocessor (μP), a microcontroller (μC), and a digital signal processor (DSP), or any combination thereof. Further, the one or more processors may include one more levels of caching, such as a level cache memory, a processor core, and registers, among other examples. The processor core may include an arithmetic logic unit (ALU), a floating point unit (FPU), and/or a digital signal processing core (DSP Core), or any combination thereof. A memory controller may be used with the processor, or, in some implementations, the memory controller may be an internal part of the memory controller. Such controllers already exist open source and closed source for purchase and additional modification.

Depending on the desired configuration, the system memory may be of any type, including, but not limited to, volatile memory (such as RAM), and/or non-volatile memory (such as ROM, flash memory, etc.), or any combination thereof. The system memory may include an operating system, one or more engines, and/or program data. In some embodiments, the engine may be an application, a software program, a service, or a software platform. The system memory may also include a storage engine that may store any information disclosed herein.

Moreover, the computing device may have additional features or functionality, and additional interfaces to facilitate communications between the basic configuration and any desired devices and interfaces, such as the position of the nozzle and communication with the one or more sensors that may be present thereon.

The computer may be used to control hardware that is not normally a part of the computing system, for example, the actuator and nozzle as disclosed herein. The computer using a computer program may control other devices such as robotic arms, and other industrial devices. The computers may also be used in one or more of data collection, as a control card for equipment, in imaging and other applications requiring high-speed data, as a controller in machine vision applications to automate quality control systems, such as the conditions in a bioreactor, be used in automatic inspection, measurement, verification, and/or flaw detection, to operate direct equipment, for example, robotic arms and in post analytics in applications that require removable drive bays for swappable hard drives for easy data backup, and/or as embedded computers.

The computer may also employ artificial intelligence (AI) or heuristic technologies. In an embodiment, the use of high speed computers allows large amounts of data to be processed and with intelligent algorithms, the software can “learn” from the data based upon patterns in causative and/or correlated data. The learning software may provide information on regarding the kinetics and thermodynamics involved with the addition of media, calcium chloride and cells to the alginate-containing container.

In an embodiment, many cell lines tend to have optimal operation and propagation at the physiological temperature of 37° C. If the temperature exceeds 38° C., the cells viability can be adversely affected. Temperatures below 37° C. result in a slower cell metabolism and slower propagation. Accordingly, in one embodiment, it is important to maintain a homogenous constant temperature in the bioreactor at 37° C. The temperature can be maintained by use of temperature sensor, with a water jacket on the bioreactor, and a temperature control unit (TCU) that adjusts the temperature so as to maintain a constant temperature.

The temperature sensor reads the actual process value of the culture medium, and consequently sends a signal to the controller to drive a change to the TCU. The TCU heats or cools down water, or any heat transfer fluid circulating in the jacket, around the bioreactor tank. The temperature of the culture media in the bioreactor equilibrates by contact with the temperature of the jacket. Additionally, the alginate solution may be temperated in the use case of printing organelles or full organs but tempering may not be necessary for printing cultivated meat structures since the product will be cold stored and/or consumed shortly after.

Similar to temperature, the certain cells demonstrate an ideal pH at which they should be grown. The use of buffered media can keep the pH in an optimal range.

Typically, many cell lines have an optimal operation at the physiological pH in the range of 7.0-7.4. A buffer solution that comprises bicarbonate buffer can be used to naturally keep the pH in that range. Other buffers that can be used include BES (N,N-bis(2-hydroxyethyl)-2-aminoethanesulfonic acid) buffer, MOPS (3-(N-morpholino) propanesulfonic acid) buffer, TES 2-[(2-Hydroxy-1,1-bis(hydroxymethyl)ethyl)amino]ethanesulfonic acid) buffer, tris buffer, phosphate buffer, glycine glycine buffer, triethanolamine buffer, or any other buffer that has a pH in or close to this physiological range. Other buffers that may be used include CO₂/HCO₃ (carbonate), phosphate, HEPES, PIPES, ACES, and TRIS. As long as a buffer has buffering capacity in the range between about pH 6 and 8, it is a candidate to be used as a buffer for the present invention.

The buffer may be associated with the container that contains the contents that are being dispersed by the nozzle or alternatively, with the container into which the contents passing through the nozzle are expelled, or both.

It should be noted that due to cellular metabolism, many cells produce CO₂ and water as they convert glucose into lactate. This causes the culture medium to become more acidic during the culture if no action is taken. In the beginning of the culture, generally the pH is regulated in the range 7.0-7.4 by playing on the bicarbonate equilibrium. If one wants to decrease the pH, generally CO₂ can be added in the sparger to increase dissolved CO₂ and decrease pH. Alternatively, if a more alkaline media is desired, air can be added in the sparger to strip the dissolved CO₂ out and increase pH. When lactate accumulates in the culture medium and if the buffering capacity of the media is exceeded, the addition of air in the sparger may be insufficient to adequately increase the pH.

Consequently, a basic solution like NaOH or Na₂CO₃ at concentrations of 0.5-1 M may be required to be introduced into the bioreactor. In one embodiment, the addition of air, CO₂ or of a basic solution may be automatically managed by a controller which compares the measurement from a pH probe inserted in the bioreactor with a defined setpoint for the process.

The cells are expanded to a target cell density using tissue culture practices in large volumes. The cells may then be differentiated or trans-differentiated into the desired cell type and consecutively transferred to the small bioreactor of the invention where they remain semi-stable until being used for printing. Cell density can be determined by any of a number of methods including the use of optical probes that do not discriminate between viable and dead cells, and/or by a radio-frequency impedance (RFI) method that counts living cells using a capacitance measure.

In order to get the cells to survive in the bioreactor, the proper media containing the appropriate nutrients should be supplied to the cells and the media may be in either the calcium chloride containing solution or in the sodium alginate containing solution (or both). Examples of the compounds that may go into the media include the buffering compounds that are described herein, deionized water, ammonium sulfate, glucose, magnesium sulfate heptahydrate, calcium chloride dihydrate, yeast extract, or other extracts.

The cell culture media according to the present invention may comprise at least one or more saccharide components, one or more amino acids, one or more vitamins or vitamin precursors, one or more growth factors, one or more salts, one or more buffer components, one or more co-factors and one or more nucleic acid components.

In an embodiment, the media may also comprise sodium pyruvate, insulin, vegetable proteins, digests or extracts, fatty acids and/or fatty acid derivatives and/or pluronic product components (block copolymers based on ethylene oxide and propylene oxide) in particular Poloxamer 188 sometimes called Pluronic F 68 or Kolliphor P 188 or Lutrol F 68 and/or surface active components like chemically prepared non-ionic surfactants. One example of a suitable non-ionic surfactant are difunctional block copolymer surfactants terminating in primary hydroxyl groups also called poloxamers.

Saccharide components may be mono- or di-saccharides, like glucose, galactose, ribose or fructose (examples of monosaccharides) or sucrose, lactose or maltose (examples of disaccharides). Saccharide components may also be oligosaccharides or polysaccharides.

In an embodiment, the media may contain amino acids such as tyrosine, the proteinogenic amino acids, especially the essential amino acids, leucine, isoleucine, lysine, methionine, phenylalanine, threonine, tryptophane and valine, as well as the non-proteinogenic amino acids like D-amino acids. The media may contain glycine. Alternatively, the media may contain alanine, arginine, asparagine, aspartic acid, cysteine, selenocysteine, glutamic acid, glutamine, glycine, histidine, isoleucine, leucine, lysine, methionine, selenomethionine, phenylalanine, proline, serine, threonine, tryptophan, tyrosine, and/or valine. Any combination of these amino acids is contemplated, and some of the amino acids may be provided in higher quantities that are necessary for the production of the cells.

In an embodiment, the media may also contain vitamins. Examples of vitamins are Vitamin A (Retinol, retinal, various retinoids, and four carotenoids), Vitamin B1 (Thiamine), Vitamin B2 (Riboflavin), Vitamin B3 (Niacin, niacinamide), Vitamin B5 (Pantothenic acid), Vitamin B6 (Pyridoxine, pyridoxamine, pyridoxal), Vitamin B7 (Biotin), Vitamin B9 (Folic acid, folinic acid), Vitamin B12 (Cyanocobalamin, hydroxycobalamin, methylcobalamin), Vitamin C (Ascorbic acid), Vitamin D (Ergocalciferol, cholecalciferol), Vitamin E (Tocopherols, tocotrienols) and Vitamin K (phylloquinone, menaquinones). Vitamin precursors and analogues are also included.

In an embodiment, the media may also contain salts. Examples of salts are components comprising inorganic ions such as bicarbonate, calcium, chloride, magnesium, phosphate, potassium and sodium or trace elements such as Co, Cu, F, Fe, Mn, Mo, Ni, Se, Si, Ni, Bi, V and Zn. Examples are copper (II) sulphate pentahydrate (CuSO₄·5H₂O), sodium chloride (NaCl), calcium chloride (CaCl₂)·2H₂O), potassium chloride (KCl), Iron (II) sulphate, sodium phosphate monobasic anhydrous (NaH₂PO₄), magnesium sulphate anhydrous (MgSO₄), sodium phosphate dibasic anhydrous (Na₂HPO₄), magnesium chloride hexahydrate (MgCl₂·6H₂O), zinc sulphate heptahydrate.

In an embodiment, the media may contain cofactors. Examples of cofactors include thiamine derivatives, biotin, vitamin C, NAD/NADP, cobalamin, vitamin B12, flavin mononucleotide and derivatives, glutathione, heme, nucleotide phosphates and derivatives.

In an embodiment, the media may contain growth factors and/or other additives.

In an embodiment, the media may contain nucleic acid components. For example, nucleic acid components, according to the present invention, may include the nucleobases, like cytosine, guanine, adenine, thymine and/or uracil, the nucleosides like cytidine, uridine, adenosine, guanosine and thymidine, and the nucleotides like adenosine monophosphate or adenosine diphosphate or adenosine triphosphate, and the corresponding nucleotides of the other nucleobases (C, U, A, T, and/or G).

In an embodiment, the media may contain the enzymes and nutrients that are involved in post expression modification for proteins grown in the cells, as well as any post-translational modifications. It should be understood that these compounds that can be added to the media may be added in any amount and at any time. For example, while the cells are in primarily an expansion state (i.e., the cells are reproducing), the media may be targeted to aid in expansion. Similarly, if the cells are in a state wherein they are not rapidly propagating but are expressing a given gene, the media may comprise more of the compounds that will aid in that process. For example, higher amounts of glycine may be introduced to the media when a collagen gene product is being expressed (due to high quantities of glycine collagen gene products).

In an embodiment, the media may contain DMEM (Dulbecco's Modified Eagle Medium), (ThermoFisher Scientific, Waltham, MA) which is a widely used basal medium for supporting the growth of many different mammalian cells. DMEM contains 4 times the concentration of amino acids and vitamins relative to the original Eagle's Minimal Essential Medium. DMEM may be formulated with low glucose (1 g/L) and sodium pyruvate, but is often used with higher glucose levels, with or without sodium pyruvate. DMEM contains no proteins, lipids, or growth factors. Therefore, in an embodiment, if DMEM is used, one will commonly supplement the DMEM with other ingredients such as 10% Fetal Bovine Serum (FBS). DMEM uses a sodium bicarbonate buffer system (3.7 g/L), and therefore requires a 5-10% CO₂ environment to maintain physiological pH. A bubbler to supply the requisite CO₂ environment may be used.

In an embodiment, a high glucose version of DMEM may be used. In the high glucose version, the DMEM contains the following amino acids, glycine, arginine, cysteine, glutamine, histidine, isoleucine, leucine, lysine, methionine, phenylalanine, serine, threonine, tryptophan, tyrosine and valine (all in the L configuration or derivatives thereof). It contains the following vitamins, choline chloride, D-calcium pantothenate, folic acid, niacinamide, pyridoxine hydrochloride, riboflavin, thiamine hydrochloride, and i-inositol. It contains the following inorganic salts, calcium chloride, ferric nitrate, magnesium sulfate, potassium chloride, sodium bicarbonate, sodium chloride, sodium phosphate monobasic. And it contains glucose and the dye, phenyl red.

In an embodiment, the techniques used during the expansion of the cells may include the usage of passage cells (i.e., cells that are grown in a media wherein removal of the media occurs with the concomitant introduction of new media into the flask or bioreactor in which the cells are being expanded).

In an embodiment, the cells that are grown in a flask, a bioreactor, or any other container in which cells can be grown, the media can be inoculated. For example, inoculation and addition ports allow the supply of nutrients, other ingredients and other media into the bioreactor/fermenter without the danger of contamination during the ongoing process. For example, by inoculating a self-sealing membrane with a sterile needle, the media is added via an inoculation or addition port.

The bioreactor may also contain other mechanical devices like slow stirrers, spargers, pH meters, pH sensors, water or other solvent jackets, other temperature regulators and sensors, filters and devices that allow for automatic expansion and/or differentiation. The expansion may take place in any type of bioreactor. For example, a continuous stirred tank bioreactor, a bubble column bioreactor, a fluidized bed bioreactor, a packed bed bioreactor, a rocker bioreactor, or an airlift bioreactor may be used, as long as they are compatible with the nozzle addition protocol and the formation of the three-dimensional alginate matrix as discussed herein. In a variation, the size of the bioreactor may be able to accommodate liquid media amounts from 1 liter to 2000 liters, or potentially bigger. In a variation, the size of the bioreactor may be between about 50 and about 2000 liters. The size of the bioreactors within the invention may be between about 100 milliliters and 10 liters. The bioreactor may have automation and data management equipment associated with it. The bioreactor may have bioprocess controllers, the requisite computer hardware and software such as the requisite computer programs and processors, and/or storage and transport equipment associated with it. The computer program may also have sensors that provide feedback to the system allowing the nozzle to move at faster or slower speeds as needed in order to form the appropriate scaffolds for growing cells, with appropriate cross-linked alginate and interface layers.

In an embodiment, the present invention relates to a system that comprises at least two containers, a first container and a second container, and a nozzle, the system comprising a calcium salt, the calcium salt being either present in the first container or introduced into the nozzle, the first container comprising components that include at least cells, one or more scaffold components, and media, the first container being operationally attached to a tube that comprises the nozzle, the tube and the nozzle designed to pass the components from the first container to the second container, wherein prior to passage of the components to the second container, the second container comprises an alginate solution. In a variation, the calcium salt is calcium chloride. In a variation, the first container comprises more than one scaffold component.

In a variation, the alginate solution forms a sodium alginate gel. In a variation, the first container and/or the second container further comprise a buffer. In a variation, the first container is under positive pressure. In a variation, the media comprises DMEM. In a variation, the nozzle is designed to move in a longitudinal, a transverse, and a vertical direction. In a variation, the nozzle is computer controlled. In a variation, the media further comprises proteins, sugars, fatty acids, vitamins, and/or minerals.

In an embodiment, the present invention relates to a method of creating a three-dimensional (3D) construct from a mixture of cells, culture medium, and a scaffold material with a calcium salt, said method comprising:
positioning a nozzle within an alginate gel solution,
moving the nozzle and depositing the mixture of cells, the culture medium, and the scaffold material in a controlled pattern within the alginate gel solution,
thereby solidifying the alginate gel solution until it forms cross-linking reactions between the calcium salt and the alginate solution, thereby resulting in formation of the three-dimensional (3D) construct comprising the mixture of cells, the culture medium, and the scaffold material.

In a variation, the calcium salt is calcium chloride. In a variation, the alginate gel solution forms a sodium alginate gel. In a variation, the culture medium comprises a buffer. In a variation, the nozzle is under positive pressure facilitating the nozzle to deposit the mixture of cells, the culture medium, and the scaffold material. In a variation, the media comprises DMEM.

In a variation, the nozzle is designed to move in a longitudinal, a transverse, and a vertical direction. In a variation, the nozzle is computer controlled. In a variation, the media further comprises one or more of proteins, sugars, fatty acids, vitamins, or minerals. In a variation, the three-dimensional (3D) construct comprises scaffolds, an interface layer and cross-linked alginate. In a variation, the first container and/or the second container is heated. In a variation, the nozzle is heated. In a variation, the first container is equipped with sensors, filters and/or actuators. In a variation, the first container comprising media, cells and one or more scaffold components is monitored. In a variation, the second container comprising the alginate solution is optionally semi-cross-linked.

In an embodiment, the present invention relates to systems and methods wherein the second container comprises the alginate solution, but may also contain additional components such as proteins like gelatine, or other polymers or polysaccharides such as pectin or cellulose. In a variation, the alginate solution may also be further functionalized by means of adding nanoparticles and/or peptides to improve cell growth and/or the mechanical/chemical properties of the gel.

It should be understood and it is contemplated and within the scope of the present invention that any feature that is enumerated above can be combined with any other feature that is enumerated above as long as those features are not incompatible. Whenever ranges are mentioned, any real number that fits within the range of that range is contemplated as an endpoint to generate subranges. In any event, the invention is defined by the below claims.

We claim:

1. A system that comprises at least two containers, a first container and a second container, and a nozzle, the system comprising a calcium salt, the calcium salt being either present in the first container or introduced into the nozzle, the first container comprising components that include at least cells, one or more scaffold components, and media, the first container being operationally attached to a tube that comprises the nozzle, the tube and the nozzle designed to pass the components from the first container to the second container, wherein prior to passage of the components to the second container, the second container comprises an alginate solution.

2. The system of claim 1, wherein the calcium salt is calcium chloride.

3. The system of claim 1, wherein the first container comprises more than one scaffold component.

4. The system of claim 1, wherein the alginate solution forms a sodium alginate gel.

5. The system of claim 1, wherein the first container and/or the second container further comprise a buffer.

6. The system of claim 1, wherein the first container is under positive pressure.

7. The system of claim 1, wherein the media comprises DMEM.

8. The system of claim 1, wherein the nozzle is designed to move in a longitudinal, a transverse, and a vertical direction.

9. The system of claim 8, wherein the nozzle is computer controlled.

10. The system of claim 1, wherein the media further comprises proteins, sugars, fatty acids, vitamins, and/or minerals.

11. A method of creating a three-dimensional (3D) construct from a mixture of cells, culture medium, and a scaffold material with a calcium salt, said method comprising positioning a nozzle within an alginate gel solution,
moving the nozzle and depositing the mixture of cells, the culture medium, and the scaffold material in a controlled pattern within the alginate gel solution,
thereby solidifying the alginate gel solution until it forms cross-linking reactions between the calcium salt and the alginate solution, thereby resulting in formation of the three-dimensional (3D) construct comprising the mixture of cells, the culture medium, and the scaffold material.

12. The method of claim 11, wherein the calcium salt is calcium chloride.

13. The method of claim 11, wherein the alginate gel solution forms a sodium alginate gel.

14. The method of claim 11, wherein the culture medium comprises a buffer.

15. The method of claim 11, wherein the nozzle is under positive pressure facilitating the nozzle to deposit the mixture of cells, the culture medium, and the scaffold material.

16. The method of claim 14, wherein the media comprises DMEM.

17. The method of claim 11, wherein the nozzle is designed to move in a longitudinal, a transverse, and a vertical direction.

18. The method of claim 17, wherein the nozzle is computer controlled.

19. The method of claim 11, wherein the media further comprises one or more of proteins, sugars, fatty acids, vitamins, or minerals.

20. The method of claim 12, wherein the three-dimensional (3D) construct comprises scaffolds, an interface layer and cross-linked alginate.

21. The system of claim 5, wherein the first container and/or the second container is heated.

22. The system of claim 1, wherein the nozzle is heated.

23. The system of claim 3, wherein the first container is equipped with sensors, filters and/or actuators.

24. The system of claim 1, wherein the first container comprising media, cells and one or more scaffold components is monitored.

25. The system of claim 1, wherein the second container comprising the alginate solution is optionally semi-cross-linked.
